(19) United States
(12) Patent Application Publication
(10) Pub. No.: US 2023/0190933 A1
(43) Pub. Date: Jun. 22, 2023

(54) Immunologically Active Peptide-Biliverdin Conjugate, Preparation Method Therefor and Application Thereof

(71) Applicant: Institute of Process Engineering, Chinese Academy of Sciences, Beijing (CN)

(72) Inventors: Xuehai Yan, Beijing (CN); Ruirui Xing, Beijing (CN); Rui Chang, Beijing (CN); Qianli Zou, Beijing (CN)

(73) Assignee: Institute of Process Engineering, Chinese Academy of Sciences, Beijing (CN)

(21) Appl. No.: 17/924,931

(22) PCT Filed: May 12, 2020
(86) PCT No.: PCT/CN2020/089886

§ 371 Date: Nov. 11, 2022

(51) Int. Cl. A61K 41/00; A61K 47/51; A61K 45/06; C07K 14/77; C07K 14/025

(52) U.S. Cl. CPC A61K 41/0057; A61K 45/06; A61K 47/51; C07K 14/025; C07K 14/77

(57) ABSTRACT

The present disclosure relates to an immunologically active peptide-biliverdin conjugate (I), a preparation method therefor and an application thereof in cancer diagnosis, and/or tumor immunotherapy, and/or tumor “photothermal immunotherapy” (tumor photothermal therapy combined with immunotherapy). The conjugate to which the present disclosure relates not only may stimulate an organism to generate a tumor-immune effect, but also may relieve and/or eliminate tumor inflammation, remodel a tumor inflammatory microenvironment and achieve photothermal cancer immunodiagnosis and immunotherapy. The conjugate to which the present disclosure relates has high biocompatibility, good stability and an extended half-life. The conjugate is prepared from an immunologically active peptide and biliverdin by means of chemical synthesis. A peptide end of the conjugate exercises the function of immunoregulation, and a pigment end thereof exercises functions such as tumor imaging diagnosis, tumor photo-thermal ablation, immune inflammatory microenvironment regulation and the like. The conjugate may significantly enhance the antitumor effect and effectively inhibit tumor metastasis and recurrence.

### FIELD

The disclosure belongs to the field of “photothermal immune” anti-tumor medicine, in particular relates to an immunologically active peptide-biliverdin conjugate, a preparation method therefor and an application thereof in cancer diagnosis, and/or tumor immunotherapy, and/or tumor “photothermal immunotherapy” (tumor photothermal therapy combined with immunotherapy).

### BACKGROUND

Traditional tumor treatment methods, such as surgery, chemotherapy, radiotherapy, etc., are faced with different degrees of side effects, and “photothermal immunotherapy” is the product of the combination of tumor photothermal therapy and tumor immunotherapy, which is a safe, accurate and broad-spectrum new treatment for tumor, shows a good therapeutic effect for metastatic and multiple-focus advanced tumor. The basic principle is that photothermal immune drugs actively targeted at, or are passively targeted at the tumor site, under the excitation of laser with specific wavelength; the drugs gathered at the tumor site absorb photothermal energy and convert it into thermal energy, which can locally raise the temperature of tumor, thus effectively killing tumor cells and inhibiting the tumor development process or clearing tumor tissues; the tumor antigen produced by photothermal therapy and the immunologically active part of photothermal immune drugs activate or enhance the immune system function, enhance the activation of immune cells and the release of immune-related factors, thus further inhibiting tumor recurrence and metastasis.

At present, various photothermal preparations, immune peptide preparations, preparation methods therefor and applications thereof have been disclosed: for example, the application of a ferrite nanomaterial in the preparation of diagnosis and treatment drugs targeted at tumor (Publication No. 106310255A); a photothermal preparation based on graphene (Publication No. CN107080844A); a controllable preparation method of selenium compounds nanosheets of copper for tumor photothermal therapy (Publication No. 106902352B); an organic micromolecule (3,6-bis (2-thienyl)-2,5-dihydropyrrolo [3,4-c] pyrrole-1,4-dione (DPP) derivative) nano tumor photothermal therapeutic reagent and preparation method thereof (Publication No. 106008525B); a nano photothermal therapeutic agent formed by polymers covalently linked through mPEG-PLGA and/or PEG-PLGA with porphyrin compounds and preparation method thereof (Publication No. 105327348A), etc. These published photothermal preparations all show high photothermal conversion efficiency and can effectively inhibit the growth of tumor, but there are still some universal problems: 1) the long-term biological safety performance needs to be studied; 2) the metabolic mechanism is not yet clear; 3) tumor metastasis and recurrence after photothermal therapy. For example, anti-tumor related peptides and related anti-cancer vaccine compositions for inducing anti-tumor immune response of colorectal cancer have been disclosed (Publication No. 103360466A); a tumor-related peptide composition and related anti-cancer vaccine for treating gliomas and other cancers have been disclosed (Publication No. 102170901A); a pharmaceutical compound (FOXP3 SIRNA-protamine-anti-CD25 antibody compound) which can enhance the anti-tumor immune response has been disclosed (Publication No. 101455840A), etc. At the same time, some immunoregulatory peptide related drugs have been commercialized, such as thymopentin for injection, recombinant human interferon a-2b injection, coriolus versicolor glycopeptide capsule, etc. However, these published or commercialized immunoregulatory drugs have the following problems: 1) small molecules with short half-life and easy degradation; 2) weak immune effect; 3) complex ingredients and serious immune-related adverse events. Therefore, it is urgent to further develop new therapeutic preparation and methods for tumor on the basis of the currently disclosed photothermal preparation and immune preparation.

Combining phototherapy with immunotherapy is expected to further inhibit tumor metastasis and recurrence on the basis of tumor ablation, and bring better survival benefits to tumor patients. Rakuten Medical developed an Antibody-Drug Conjugates (ADC) composed of cetuximab and IRDye700DX, which is used for the combination of photodynamic therapy and immunotherapy, specifically, cetuximab-mediated targeted delivery is used to achieve high tumor specificity, and IRDye700DX photodynamic effect is used to achieve tumor ablation. At present, in the clinical trials for locally recurrent head and neck cancer, this technique has achieved a therapeutic effect of 50% remission rate, 16.7% complete remission rate and 86.7% disease control rate, and good biological safety. However, its complex structure has caused difficulties in synthesis and production to some extent. At the same time, photodynamic therapy is strongly dependent on oxygen which is therefore not suitable for hypoxic tumor. The corresponding tumor “photothermal immunotherapy” has shown remarkable advantages, especially its broad spectrum and accuracy. The success of this therapy mainly depends on the photo-thermal conversion efficiency of “photothermal immune” drugs and the immune effect. At the same time, the biological safety, immune-related adverse events (irAEs) and metabolic mechanism of “photothermal immune” preparations are also the keys to the successful implementation of tumor photothermal immunotherapy.

Endogenous pigment biliverdin, a secondary metabolite of hemoglobin in animals, has a clear metabolic mechanism and multiple biological activities (antioxidant, anti-inflammatory, anti-tumor, etc.). Biliverdin is a bioactive pigment with linear tetrapyrrole structure, which has remarkable near-infrared absorption and can effectively convert near-infrared light into thermal energy (Patent Publication No. 109224073A), and has a broad application prospect in the development of tumor therapeutic drugs and photothermal anti-tumor fields. Studies have proved that tumor inflammatory microenvironment can further promote tumor metastasis and recurrence, but it has not been publicly reported that the anti-inflammatory activity of biliverdin molecule can be used to reverse tumor inflammatory microenvironment and achieve tumor immunotherapy. At the same time, small-molecule immunologically active peptides are substances with specific amino acid sequences and multiple biological functions (immunoregulation, anti-tumor, etc.), and gradually show unique advantages in the biomedical field.

There is no public report on the conjugate formed by biliverdin molecule and immunologically active peptide molecule by means of chemical synthesis, preparation method therefor and application thereof in tumor “photothermal immunotherapy”. An immunologically active peptide-biliverdin conjugate disclosed in this application has remarkable advantages in the field of cancer diagnosis, and/or tumor immunotherapy, and/or tumor “photothermal immunotherapy”: 1) an extended half-life and enhanced stability, compared with the disclosed immunologically active peptide and composition thereof; 2) high biological safety, single component and clear metabolism mechanism in vivo; 3) an immunologically active peptide end which can stimulate an organism to generate a tumor-immune response and enhance immune function; the biliverdin end can achieve cancer diagnosis and photothermal treatment, relieve and eliminate tumor inflammation, and remodel the tumor inflammatory microenvironment. In summary, the molecular conjugate of the present disclosure and related preparations, dosage forms thereof and preparation methods thereof are of great significance in promoting clinical application in the treatment of tumor, and have great application potential in eliminating primary tumor, inhibiting tumor metastasis and recurrence, etc.

### SUMMARY

The disclosure discloses an immunologically active peptide-biliverdin conjugate, a preparation method therefor and an application thereof in tumor imaging, tumor immunotherapy and tumor “photothermal immunotherapy”. The disclosed conjugate has the following advantages: 1) an extended half-life and enhanced stability, compared with the disclosed immunologically active peptide and composition thereof; 2) high biological safety, single component and clear metabolism mechanism in vivo; 3) An immunologically active peptide end which can stimulate an organism to generate a tumor-immune response and enhance immune function; the biliverdin end can realize tumor multi-mode imaging and tumor photothermal treatment, achieve tumor ablation, and also can relieve and eliminate tumor inflammation, remodel the tumor inflammatory microenvironment, and prevent tumor metastasis and recurrence.

To achieve the purpose of the disclosure, the disclosure adopts the following technical solution:
on the first aspect, the present disclosure provides an immunologically active peptide-biliverdin conjugate, which is characterized in that the structure follows formula i or ii or iii, and the salts, isomers and derivatives that do not affect pharmaceutical function thereof: (i)

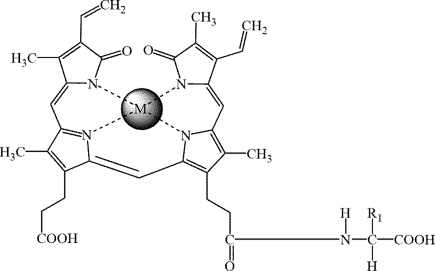

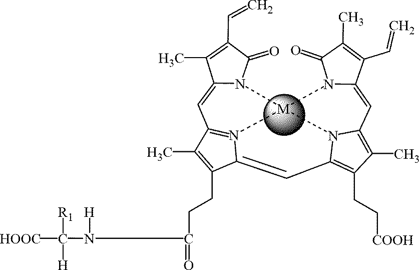

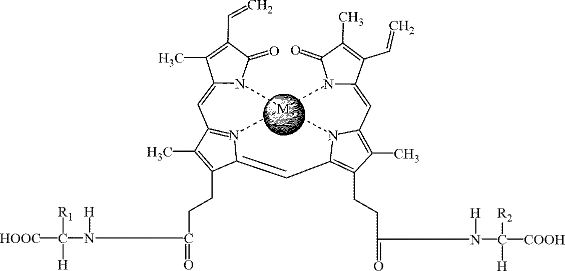

wherein,
M is selected from the following nonmetallic atoms or ions of nonmetallic elements: H, Si, P, or metallic atoms or ions of metallic elements: Mg, Al, Cr, Mn, Fe, Co, Ni, Cu, Zn, Ga, Ge, Y, Ru, Rh, Pd, In, Sn, Pt, Au, Eu, Gd, Tb, Dy, Er, Yb, Lu, Tc, Tl, radioisotopes and nonradioactive isotopes thereof; The number of M is 1-4, and the specific number varies with the valence of M;
R₁ and R₂ respectively represent an active peptide with the function of immunoregulation, and its amino acid sequence is any one of any group of X₁-X₂₂:
   X₁: Ovalbumin peptide: SIINFEKL (8), EQLESIINFEKLTE (14), ISQAVHAAHAEINEAGR (17)
   X₂: HPV16 E7 peptide: PDRAHYNI (8), TLGIVCPI (8), RAHYNIVTF (9), YMLDLQPETT (10), GQAEPDRAHYNIVTF (15)
   X₃: NYSKPTDRQYHF (12), KHAHHTHNLRLP (12)
   X₄: HVIHEGTVVI (10), HVVHEGTVVI (10)
   X₅: KVPRNQDWL (9), FLWGPRALV (9)
   X₆: YLEPGPVTA (9), IMDQVPFSV (9)
   X₇: MLLAVLYCL (9), YMDGTMSQV (9)
   X₈: TKPR (4)
   X₉: GQPR (4)
   X₁₀: CAPE (4)
   X₁₁: RKEVY (5)
   X₁₂: RKDVY (5) X₁₃: LVVTPW (6)
   X₁₄: FLGFPT (6)
   X₁₅: PDRAHYNI (8)
   X₁₆: FKFEFKFE (8)
   X₁₇: ALCNTDSPL (9)
   X₁₈: KIFGSLAFL (9)
   X₁₉: KTKCKFLKKC (10)
   X₂₀: QQKFQFQFEQQ (11)
   X₂₁: PLYKKIIKKLLES (13)
   X₂₂: HSLGKWLGHPDKF (13)
   X₂₃: VHFFKNIVTPRTP (13)
   X₂₄: EIIVTHFPFDEQNCSMK (17)
   X₂₅: (SNTSESF)2KFRVTQ-LAPKQIKE-NH₂ (29).

On the second aspect, the present disclosure provides the conjugate according to the first aspect, characterized in that R₁ and R₂ are the same or different.

On the third aspect, the present disclosure provides the conjugate according to the first to second aspects, characterized in that R₁ and R₂ are any sequences of the above, also can be peptides or protein comprising any sequences of the above, or derivatives of any sequences of the above, or amino acids, peptides or protein with similar functions;
preferably, it is characterized in that the active site of the immunologically active peptide is at the non-N end, and the inactive end is condensed with the C end of biliverdin by peptide bond;
further preferably, it is characterized in that the immunologically active peptide has the following amino acid sequence, or comprises the following sequence, or is a derivative of the following sequence, or is an amino acid, peptide or protein with similar functions:
   X₁: SIINFEKL (8)
   X₃: NYSKPTDRQYHF (12)
   X₅: FLWGPRALV (9)
   X₆: YLEPGPVTA (9), IMDQVPFSV (9)
   X₇: YMDGTMSQV (9)
   X₁₅: PDRAHYNI (8)
   X₁₈: KIFGSLAFL (9)
   X₂₃: VHFFKNIVTPRTP (13)
wherein, the derivative is a peptide molecule or a key molecule fragment thereof modified by phenyl, benzyloxycarbonyl, tert-butoxycarbonyl, beta-naphthyl-amido, N-(3-indolacetyl) or N-fluorene methoxycarbonyl groups.

On the fourth aspect, the present disclosure provides the conjugate according to the first to third aspects, characterized in that involving molecular conjugates and preparations or dosage forms derived from the molecular conjugates:
comprising: preparations or dosage forms system formed by chemical bonding, physical adsorption, loading or wrapping; and assemblies, polymers or aggregates formed by weak intermolecular interaction;
wherein, the preparations or dosage forms comprise solution, emulsion, suspension, tablet, gel or patch.

On the fifth aspect, the present disclosure provides the method for preparing the conjugate according to the first to fourth aspects, characterized in that comprising the following steps:
(1)M is H:
   a. adding biliverdin, 1-ethyl-(3-dimethylaminopropyl) carbodiimide hydrochloride (EDC•HCl), N-hydroxysuccinimide (NHS) and anhydrous dimethylformamide (DMF) into a reactor sequentially, and mixing uniformly;
      the concentration of the biliverdin is 0.1-500 mM, preferably, the concentration is 1-100 mM;
      the concentration of the EDC-HCI is 0.1-1000 mM, preferably, the concentration is 1-200 mM;
      the concentration of NHS is 0.1-1000 mM, preferably, the concentration is 1-200 mM;
      the mass concentration ratio of biliverdin, EDC•HCl and NHS is 1: 1: 0.5-1: 20: 20, preferably, the mass concentration ratio is 1: 1: 0.8-1: 5: 10;
   b. stirring the mixture obtained in step (a) at room temperature in the dark for 12-48 h, preferably 12-24 h;
   c. adding water to the mixture obtained in step (b) while stirring, and collecting the precipitate;
   d. adding anhydrous DMF into the precipitate obtained in step (c), wherein the mass ratio of the precipitate and DMF is 1:100, preferably 1: 5;
   e. adding immunologically active peptide and anhydrous triethylamine into the anhydrous DMF solution of the precipitate obtained in step (d), and stirring at room temperature in the dark;
      the concentration of the immunologically active peptide is 0.01-2000 mM, preferably, the concentration is 0.1-500 mM;
      the concentration of anhydrous triethylamine is 0.01-4000 mM, preferably, the concentration is 0.1-1,000 mM;
      the stirring time is 4-96 h, preferably 12-24 h;
   f. adjusting the pH value of the mixed solution obtained in step (e) to 3.5-7.5, preferably, the pH value is 4.0-6.0;
      the pH value is adjusted by adding alkaline or acidic substances:
         preferably, the alkaline substance is any one or a mixture of two or more of sodium hydroxide, potassium hydroxide and sodium carbonate;
         preferably, the acidic substance is any one or a mixture of two or more of hydrochloric acid, sulfuric acid and nitric acid;
         the pH value can also be adjusted by an aqueous solution dialysis method;
   g. collecting the precipitate in step (f) and purifying by size exclusion chromatography;
   h. recrystallizing the substance obtained in step (g) to obtain a pure molecular conjugate.
(2)M is a metal atom or ion except H:
   i. dissolving biliverdin and excess metal acetate in methanol, wherein the mass concentration ratio of biliverdin and metal salt is 1: 1-1: 100, preferably 1: 2-1: 20;
   j. stirring the methanol solution in step (i) at a certain temperature for 4 hours, with the temperature ranging from 20° C. to 60° C., preferably from 35° C. to 60° C.;
   k. removing the solvent from the solution obtained in the step (j) by rotary evaporation to obtain a solid;
   l. purifying the solid obtained in the step (k) by a reversed-phase chromatographic column to obtain the biliverdin-metal complex;
   m. according to steps a to h, the synthesis of biliverdin-metal complex-immunologically active peptide conjugate (conjugate of biliverdin-metal complex and immunologically active peptide) is achieved.
(3)M is a nonmetallic atom or ion except H:
   n. dissolving the biliverdin and nonmetallic halide or acyl chloride in organic solvent pyridine or DMF, wherein the mass concentration ratio of biliverdin and nonmetallic halide or acyl chloride is 1: 1-1: 100, preferably 1: 2-1: 20;
   o. stirring the mixed solution obtained in step (14) at a certain temperature in the dark, with the temperature ranging from 20 to 100° C., preferably from 35 to 65° C.; the reaction time is 2-8 h, preferably 4-6 h;
   p. removing the solvent from the solution obtained in the step (15) by rotary evaporation;
   q. purifying the solid obtained in the step (16) by a reversed-phase chromatographic column to obtain the biliverdin-nonmetal complex;
   r. according to steps a to h, the synthesis of biliverdin-nonmetal complex-immunologically active peptide conjugate (conjugate of biliverdin-metal complex and immunologically active peptide) is achieved.

On the sixth aspect, the present disclosure provides the conjugate according to the first to fifth aspects and the preparation method thereof, which is characterized in that the conjugate has “photothermal immune” anti-tumor use.

On the seventh aspect, the present disclosure provides the use according to the sixth aspect, which is characterized in that the conjugate molecules gathered in the tumor position can complete the conversion of light energy to thermal energy under the irradiation of laser with specific wavelength, and therefore realize tumor ablation, and at the same time, after illumination the tumor position can generate in-situ tumor specific antigen; the conjugate can further activate the immune response of the organism, eliminate tumor inflammatory microenvironment, enhance the specific immune response of the organism, thus achieving immuno-treatment of tumor and further preventing tumor metastasis and recurrence. That is, the combination of tumor photothermal therapy and tumor immunotherapy is achieved, which is characterized in that the combination of tumor ablation, immunoregulation and tumor inflammatory microenvironment regulation is achieved, and the treatment effect of tumor is obviously improved; wherein
the laser wavelength of the tumor ablation is 635 nm, 660 nm, 680 nm, 730 nm, 808 nm, 980 nm and 1064 nm, preferably 730 nm and 808 nm; the laser intensity is 0.05-2.5 W/cm², preferably, the laser intensity is 0.2-1.2 W/cm²;
the tumor immunoregulatory effect is one effect from, or synergy of two or more effects from the enhancement of antigen recognition, uptake and presentation; improvement of activation, proliferation and differentiation of immune cells; increase of immune cytokines secretion;
the effect of tumor inflammatory microenvironment regulation is one effect from, or synergy of two or more effects from inhibiting the effect of inflammatory cells, inhibiting secretion of inflammatory related factors and blocking intracellular signal pathway.

On the eighth aspect, the present disclosure provides the use according to the sixth to seventh aspects, characterized in that the conjugate is used for tumor diagnosis and monitoring before, during and after “photothermal immunotherapy”, comprising nuclear magnetic resonance imaging, radionuclide imaging and photoacoustic imaging.

On the ninth aspect, the present disclosure provides the use according to the eighth aspect, characterized in that the conjugate is used for nuclear magnetic resonance imaging of tumor, and M is preferably selected from the following atoms or ions: Mn, Fe, Cu, Eu, Gd, Dy.

On the tenth aspect, the present disclosure provides the use according to the eighth aspect, characterized in that the conjugate is used for radionuclide imaging of tumor, and M is preferably selected from the following atoms or ions: ^{64,67}Cu, ⁹⁹mTc, ¹⁹⁵Pt, ^{67,68}Gd, ²⁰¹Tl, ⁶⁰Co, ¹¹¹In and ⁵¹Cr.

On the eleventh aspect, the present disclosure provides the use according to the eighth aspect, characterized in that the conjugate is used for photoacoustic imaging of tumor, and M is preferably selected from the following atoms or ions: H and Zn.

On the twelfth aspect, the present disclosure provides the use according to the sixth to eleventh aspects, characterized in that the tumor is a primary tumor or a metastatic tumor, and is selected from single or multiple tumors such as brain cancer, head and neck cancer, esophageal cancer, breast cancer, lung cancer, stomach cancer, liver cancer, colon cancer, pancreatic cancer, lymphoma, melanoma, ovarian cancer, cervical cancer, prostate cancer and bladder cancer. Preferably, the tumor is a superficial tumor or a tumor with high surgical risk; for example, the superficial tumor or the tumor with high surgical risk comprising head and neck cancer, breast cancer, melanoma, cervical cancer, prostate cancer, pancreatic cancer and et al.

On the thirteenth aspect, the present disclosure provides the use according to the sixth to eleventh aspects, which is characterized in that it can be combined with tumor therapy strategies such as surgery, chemotherapy, radiotherapy and immunotherapy.

On the fourteenth aspect, the present disclosure provides the use according to the thirteenth aspect, which is characterized in that it is used for the treatment after postoperative of residual tumor lesions and/or metastatic tumor lesions.

On the fifteenth aspect, the present disclosure provides the use according to the thirteenth aspect, which is characterized in that it is used for the combination of chemotherapy and “photothermal immunotherapy”;
wherein, the chemotherapy drugs used comprise at least one selected from the group consisting of: cisplatin, carboplatin, nedaplatin, oxaliplatin, lobaplatin, carmustine, lomustine, smoustine, nimustine, methotrexate, pemetrexed, nolatrexed, raltitrexed, fluorouracil, capecitabine, gemcitabine, ancitabine, cytarabine, tegafur, fluorouridine, doxifluridine, youfuding, vinblastine, vincristine, vinblastine, vindesine, vinorelbine, paclitaxel, docetaxel, albumin-bound paclitaxel, camptothecin, irinotecan, topotecan, rubitecan, doxorubicin (adriamycin), epirubicin, pirarubicin, amide, isocyclophosphamide and etoposide, or derivatives thereof, preferably cisplatin, paclitaxel, docetaxel and doxorubicin (adriamycin).
preferably, the dosage of chemotherapeutic drugs is 5%-30% of the conventional dosage, and further preferably 10%-15%.

On the sixteenth aspect, the present disclosure provides the use according to the thirteenth aspect, which is characterized in that it is used for the combination of radiotherapy and “photothermal immunotherapy”;

preferably, the dose of radiation is 5%-40% of the conventional dose, and further preferably, 5%-20%.

On the seventeenth aspect, the present disclosure provides the use according to the thirteenth aspect, which is characterized in that it is used for the combination of immunotherapy and “photothermal immunotherapy”;
preferably, the immunotherapy drugs comprise antibodies, cytokines, molecular vaccines, cell vaccines, biological response regulators, immune inhibitor, and monomer components of traditional Chinese medicine;
further preferably, the immunotherapy drugs comprise thymic factor, indoleamine 2,3-dioxygenase inhibitor, interferon and interleukin;
further preferably, the dose of the immune drug is 10%-50% of the conventional dose, still further preferably 15%-30%.

On the eighteenth aspect, the conjugate according to the first to seventeenth aspects, the preparation method therefor and application thereof in tumor therapy provided by the present disclosure have the advantages of high biological safety, good stability, difficulty to develop drug resistance, definite metabolic mechanism, extended half-life and the like, and can significantly enhance the anti-tumor effect.

### Compared With the Prior Art, the Disclosure Has at Least the Following Beneficial Effects

1) At present, there are no reports of “photothermal immune” molecules, preparations and dosage forms based on biliverdin, and no related reports of its effects of tumor immunotherapy and/or “photothermal immunotherapy”, in particular, the conjugate of the disclosure can improve the photothermal effect of biliverdin;

2) An immunologically active peptide-biliverdin molecular conjugate, which is obtained by chemical synthesis from endogenous biliverdin and immunologically active peptide, has high biological safety, good stability and clear metabolic mechanism, and can effectively solve the problems of serious immune-related adverse events (irAEs) and poor biocompatibility.

3) Multi-functional synergy can be achieved in tumor therapy: the immunologically active peptide end can stimulate the organism to generate tumor immune response and enhance immune function; the biliverdin end can realize tumor imaging, tumor photothermal treatment and tumor ablation, and also can relieve and eliminate tumor inflammation, remodel the tumor inflammatory microenvironment, and reduce tumor metastasis rate and recurrence rate.

### BRIEF DESCRIPTION OF THE DRAWINGS

FIG. 1 is the molecular structure diagram of the conjugate (a) prepared in Example 1, and the cyclic (three) photothermal heating curves of biliverdin molecule (b) and conjugate molecule (1c), indicating that the conjugate molecule has better heating effect and better cyclic stability.

FIG. 2 is the cell activity test result of the conjugate prepared in Example 2, demonstrating that the prepared conjugate has high biological safety and no obvious cytotoxicity to Human Umbilical Vein Endothelial Cells HUVEC.

FIG. 3 is the cell activity test result of the conjugate prepared in Example 4, demonstrating that the prepared conjugate has high biological safety and no obvious cytotoxicity to mouse skin melanoma cells B 16-F 10.

FIG. 4 is the relative fluorescence intensity of positive BMDCs at different time points in Example 4, indicating that BMDCs can successfully uptake this conjugate, laying a foundation for further tumor immunotherapy.

FIG. 5 is the promoting effect of the conjugate prepared in Example 5 on the maturation of dendritic cells, indicating that the conjugate molecule can promote the maturation of dendritic cells, laying a foundation for further tumor immunotherapy;

FIG. 6 is the specific binding results of the conjugate (FITC labeled) prepared in Example 6 with DU-145 cell line and LNCaP cell line.

FIG. 7 is the temperature rise in vitro of the conjugate obtained in Example 7 under laser irradiation, showing that the conjugate molecules have good photo-thermal conversion effect, laying a foundation for “photothermal immunotherapy” of tumor.

FIG. 8 is the anti-tumor behavior of the conjugate in Example 8 in the absence of light, indicating that the conjugate molecule has potential immune anti-tumor activity.

FIG. 9 is the tumor inhibition curve (a) and recurrence curve (b) of the conjugate in Example 9, indicating that the conjugate has a good tumor “photothermal immunotherapy” effect and can effectively prevent tumor recurrence.

FIG. 10 is the content of immune-related factors in Example 10, indicating that the conjugate can up-regulate the organism’s immunity and down-regulate the immunosuppressive behavior with the presence and absence of light, indicating that the conjugate has a tumor “photothermal immunotherapy” effect.

FIG. 11 is the transmission electron microscope picture of the conjugate molecular gel in Example 11, showing a regular fiber network structure.

FIG. 12 is the statistical chart of the results of the conjugate emulsion type in Example 12 used for tumor imaging of mouse bladder cancer, confirming the cancer diagnosis ability of the conjugate.

FIG. 13 is the curve of CD4⁺T and CD8⁺T cells in spleen and draining lymph nodes of mice under the action of the conjugate in Example 13, which shows that the conjugate has immune effect and “photothermal immune” effect.

FIG. 14 is the tumor radionuclide imaging results (a), in-situ tumor temperature rise (b) of the conjugate in Example 14, and the expression of CD8⁺T cells (c) and CD107 molecules on the surface of CD8⁺T cells (d) in mouse tumors, which proves that the conjugate can be used for cancer diagnosis and tumor “photothermal immunotherapy”.

FIG. 15 is the combined inhibitory effect of conjugate of Example 15 combined with immune preparation on B16-F10 tumor and Lewis tumor, showing that the combination of “photothermal immunity” and immunotherapy significantly enhances the anti-tumor effect.

FIG. 16 is the combined inhibitory effect of the conjugate of Example 16 combined with low-dose chemotherapy drugs on B16-F10 tumor and Lewis tumor, showing that the combination of “photothermal immunity” and chemotherapy can significantly enhance the anti-tumor effect (a) and effectively reduce the influence on the survival state (body weight, b) of mice.

FIG. 17 is the influence of the conjugate in Example 17 on important organs (heart, liver, spleen, lung, kidney), showing that it does not cause serious damage to important organs.

### DETAILED DESCRIPTION OF THE EMBODIMENTS

To further explain the technical solution and effects of the present disclosure, the technical solution of the present disclosure will be further explained below with reference to the preferred embodiments of the present disclosure, but the present disclosure is not limited to the scope of the embodiments.

The embodiments without specific technology or conditions are carried out according to the technology or conditions described in the literature in the field or according to the product description. The reagents or instruments used without indicating the manufacturer are conventional products that can be purchased through regular channels.

### Example 1

According to the following steps, the biliverdin-SIINFEKL conjugate is obtained through chemical synthesis: certain amounts of biliverdin, EDC-HCl, NHS and DMF were weighed, sequentially added into a reactor and mixed uniformly; the obtained mixture was stirred at room temperature in the dark for 24 h; water was added and stirred, and precipitate was collected; anhydrous DMF was added into the obtained precipitate, and the mixture was mixed uniformly, then SIINFEKL peptide and anhydrous triethylamine were added and stirred, and these substances reacted at room temperature in the dark for 24 h; the precipitate of the above reaction was collected, and purified by size exclusion chromatography; the obtained substance was recrystallized to obtain a pure molecular conjugate. Wherein, the concentration of biliverdin was 100 mM, the concentration of EDC. HCI was 100 mM, the concentration of NHS was 50 mM and the concentration of peptide was 200 mM. 1H NMR information of the prepared conjugate was as follows:

1H NMR (600 MHz) 6 = 11.95 - 13.00 (3H), 10.68 (s, 1H), 8.85 (s, 1H), 8.56 (s, 1H), 8.40 (s, 1H), 8.32 (s, 2H), 8.21 (s, 2H), 8.08 (s, 1H), 7.50 (s, 1H), 7.41 (s, 1H), 7.30 (m, 3H), 7.16 (m, 3H), 7.03 (q, 2H), 6.93 (s, 1H), 6.49 (t, 2H), 5.57 (s, 1H), 5.20 (m, 4H), 4.16 (m, 1H), 3.95 (m, 1H), 4.55 (m, 2H), 4.92 (m, 1H), 4.34 (m, 2H), 4.94 (m, 1H), 4.84 (m, 1H), 4.61 (1H), 4.44 (m, 2H), 3.44 (m, 1H), 3.18 (m, 1H), 2.81 (m, 1H), 2.69 (m, 4H), 2.49 - 2.42 (7H), 2.35 (m, 3H), 2.23 (m, 2H), 2.12 (m, 6H), 2.06 (m, 2H), 1.95 (m, 3H), 1.49 (m, 1H), 1.75 (m, 4H), 1.55 (m, 6H), 1.25 (m, 2H), 1.11 (m, 6H), 0.90 - 1.00 (12 H).

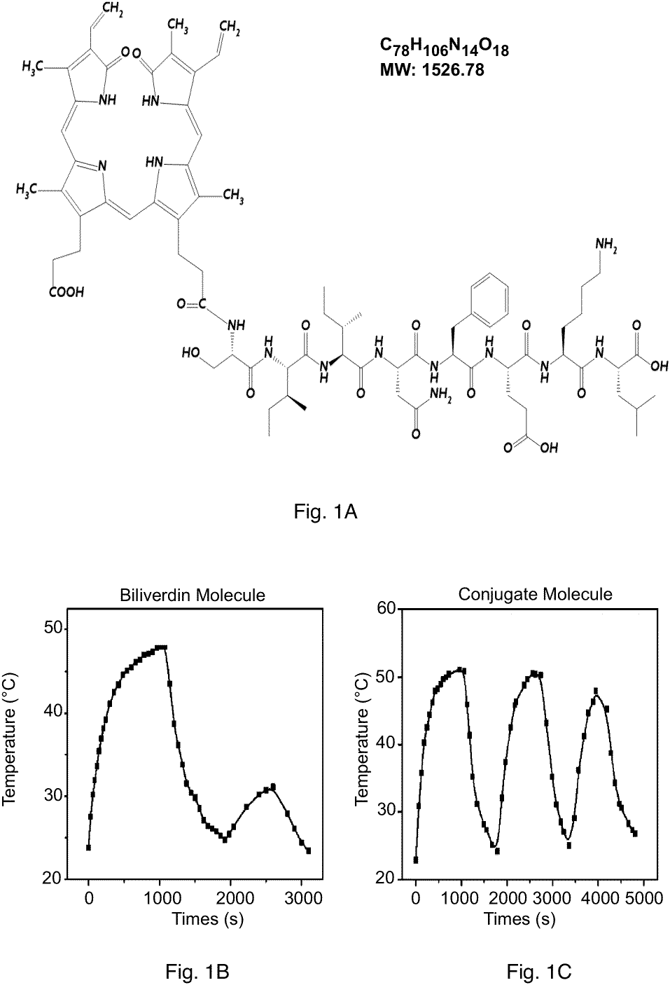

FIG. 1(a) is the molecular structure diagram of the conjugate prepared in Example 1, and FIG. 1(b) is the cyclic (three) photothermal heating curves of biliverdin molecule and conjugate molecule (FIG. 1c), which indicates that the conjugate molecule has better heating effect and better cyclic stability.

### Example 2

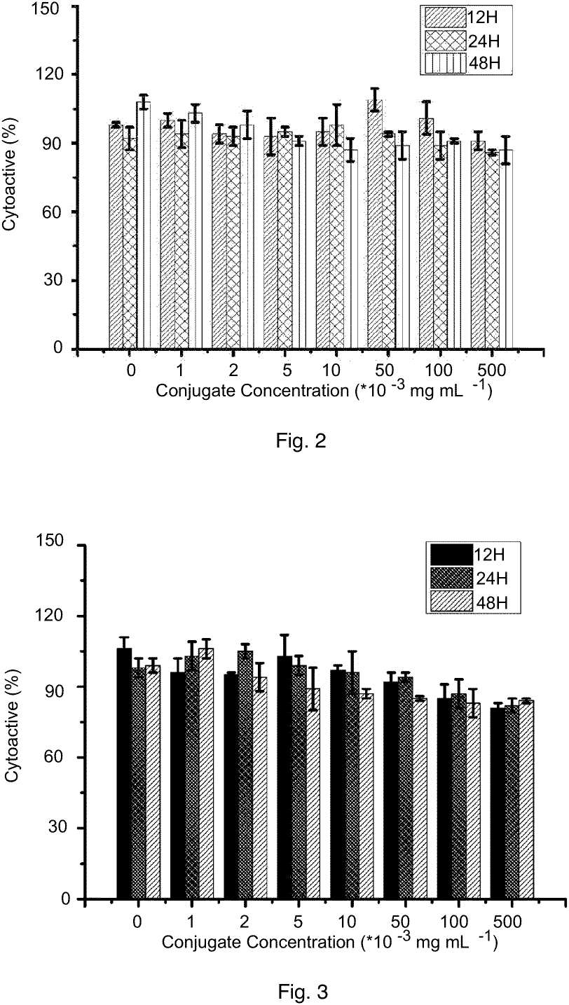

The biliverdin-SIINFEKL conjugate was prepared from biliverdin molecule and SIINFEKL according to the chemical synthesis method of Example 1. A certain amount of conjugate was weighed, after being pre-dissolved in trace DMSO solution, the conjugate was directly dissolve in PBS solution, filtration sterilization was conducted after stirring and dissolving, and the pH value was adjusted to neutral. The prepared conjugates with different concentration gradients were incubated with Human Umbilical Vein Endothelial Cells HUVEC in the dark, and the biological safety of the conjugates was evaluated by MTT colorimetric method. FIG. 2 is the cell activity test results of the conjugate prepared in Example 2, which shows that the prepared conjugate has high biological safety and no obvious cytotoxicity to Human Umbilical Vein Endothelial Cells HUVEC.

### Example 3

Firstly, biliverdin was chemically synthesized with excess zinc acetate to obtain biliverdin-Zn metal complex, and the experimental method was as follows: biliverdin and excess zinc acetate were dissolved in methanol solution, stirred at 60° C. for 4 hours, solid was obtained by removing solvent from the obtained solution by rotary evaporation, the solid wad then purifed by reversed-phase chromatographic column to obtain biliverdin-Zn complex, wherein the mass concentration ratio of biliverdin and zinc acetate is 1:5. The biliverdin-Zn-NYSKPTDRQYHF conjugate was prepared from biliverdin-Zn metal complex and NYSKPTDRQYHF according to the above chemical synthesis method. A certain amount of conjugate was weighed, after being pre-dissolved in trace DMSO solution, the conjugate was directly dissolve in PBS solution, filtration sterilization was conducted after stirring and dissolving, and the pH value was adjust to neutral. The prepared conjugates with different concentration gradients were incubated with mouse skin melanoma cells B16-F10 in the dark, and the biological safety of the conjugates was evaluated by MTT colorimetric method. FIG. 3 is the cell activity test results of the conjugate prepared in Example 3, which shows that the prepared conjugate has high biological safety and no obvious cytotoxicity to mouse skin melanoma cells B 16-F10.

### Example 4

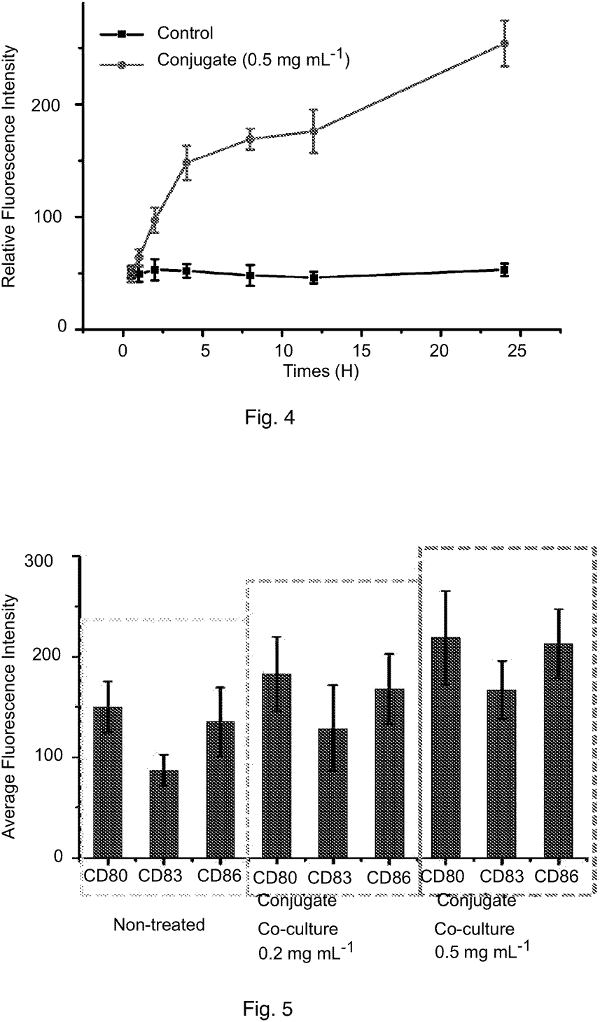

Firstly, biliverdin was chemically synthesized with excess ferrous chloride to obtain biliverdin-Fe metal complex, and the biliverdin-Fe-YMDGTMSQV conjugate was prepared from biliverdin-Fe metal complex and YMDGTMSQV according to the aforementioned chemical synthesis method. A certain amount of conjugate was weighted, after being pre-dissolved in a small amount of organic solvent, the conjugate was completely dissolved in PBS solution, filtration sterilization was conducted, and the pH value was adjusted to neutral. Fluorescein was used to label the conjugate, and the labeled conjugate was incubated with mouse Bone Marrow-derived Dendritic Cells BMDCs, the uptake of the conjugate by BMDCs was detected by flow cytometry. FIG. 4 shows the relative fluorescence intensity of positive BMDCs at different time points in Example 4, which indicates that BMDCs can successfully uptake this conjugate, laying a foundation for further tumor immunotherapy.

### Example 5

The biliverdin-KIFGSLAFL conjugate was prepared from biliverdin molecule and KIFGSLAFL according to the above chemical synthesis method. A certain amount of conjugate was weighed, after being pre-dissolving in a small amount of organic solvent, the conjugate was completely dissolve in PBS solution, filtration sterilization was conducted, and the pH value was adjusted to neutral. The prepared conjugate molecular solution was cocultured with dendritic cells from peripheral blood of non-small cell lung cancer model mice, after 24 hours, the dendritic cells were collected, washed and fluorescently labeled, and the CD80, CD83 and CD86 on the cell surface were detected by flow cytometry to evaluate the promoting effect of the conjugate on the maturation of dendritic cells. FIG. 5 shows the promoting effect of the conjugate prepared in Example 5 on the maturation of dendritic cells, showing that the conjugate molecule can promote the maturation of dendritic cells, which lays a foundation for further tumor immunotherapy.

### Example 6

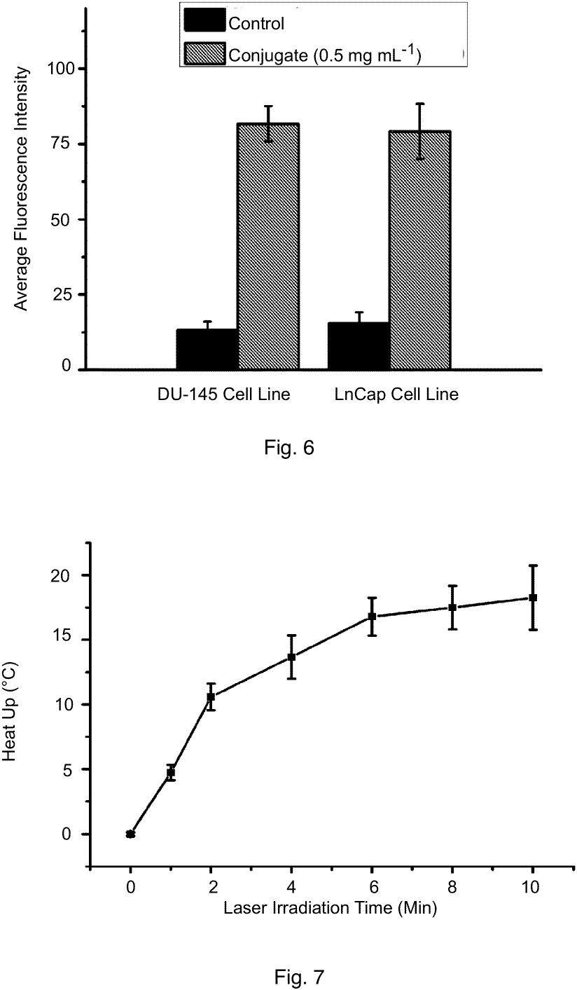

The biliverdin-FLWGPRALV conjugate was prepared from biliverdin molecule and FLWGPRALV according to the above chemical synthesis method. A certain amount of conjugate was weighed, directly dissolved in PBS solution, filtration sterilization was conducted after stirring and dissolving, and the pH value was adjusted to neutral. The prepared conjugate was co-incubated with human prostate cancer DU-145 cell line and LNCaP cell line, and analyzed by flow cytometry. The data were statistically analyzed by SPSS 12.0. FIG. 6 shows the specific binding results of the conjugate (FITC labeled) prepared in Example 6 with DU-145 cell line and LNCaP cell line.

### Example 7

Firstly, biliverdin-Mn metal complex was chemically synthesized by biliverdin and excess manganese acetate tetrahydrate, and the biliverdin-Mn-YLEPGPVTA conjugate was prepared from biliverdin-Mn metal complex and YLEPGPVTA according to the aforementioned chemical synthesis method. A certain amount of conjugate was weighed, after being pre-dissolved in a small amount of organic solvent, the conjugate was completely dissolved in PBS solution, filtration sterilization was conducted, and the pH value was adjusted to neutral. 1 mL of this conjugate (at a concentration of 0.2 mg mL⁻¹) was irradiated at 730 nm laser (0.3 W/cm²) for 10 min, and the temperature rise of the conjugate solution was investigated. FIG. 7 shows the temperature rise in vitro of the conjugate obtained in Example 7, showing that the conjugate molecule has good photo-thermal conversion effect, which lays a foundation for the realization of tumor “photothermal immunotherapy”.

### Example 8

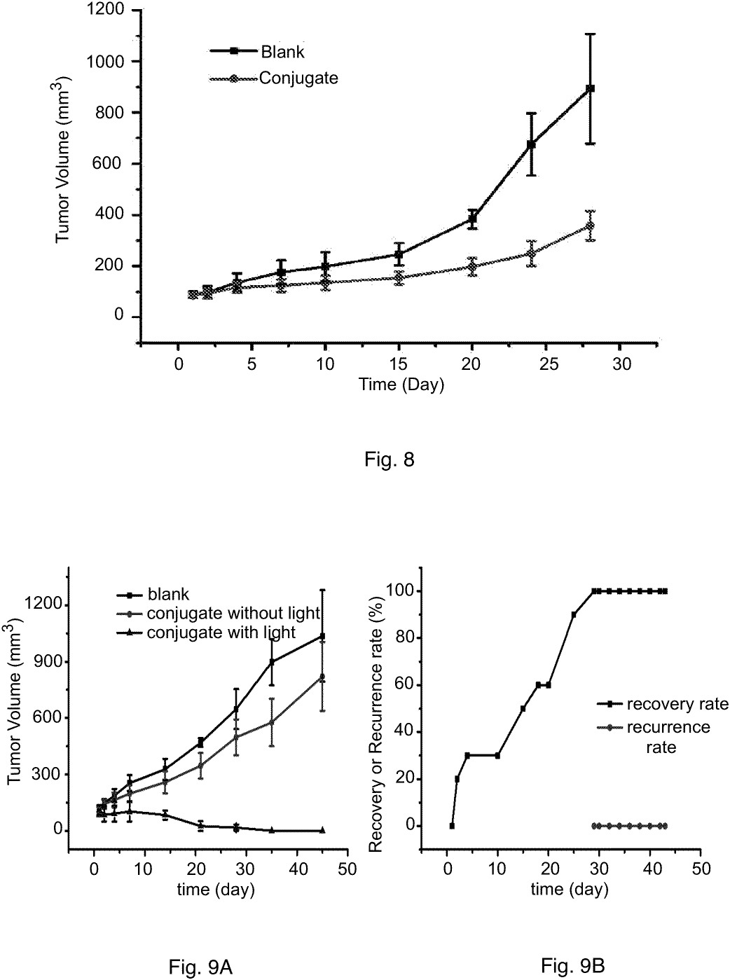

The biliverdin-IMDQVPFSV conjugate was prepared from biliverdin molecule and IMDQVPFSV according to the above chemical synthesis method. A certain amount of conjugate was weighted , after being pre-dissolved in trace DMSO solution, the conjugate was directly dissolved in PBS solution, filtration sterilization was conducted after stirring and dissolving, and the pH value was adjusted to neutral. According to the standard tumor mouse modeling method, C57BL/6 mouse model was established, and the mouse breast cancer cells 4T1 were inoculated subcutaneously, and then these mice were fed in SPF environment, the tumor growth was observed at any time, and relevant experiments were carried out after the average tumor volume reached about 80-100 mm³. The mice were divided into two groups (10 mice in each group), on the 1st, 2nd, 4th and 8th day, the mice in experimental group was injected with 100 uL of the conjugate (at a concentration of 0.2 mg mL⁻¹) intraperitoneally, while the mice in blank group was injected with the same quality of normal saline. The growth of tumor volume in mice was monitored within 28 days. FIG. 8 shows the anti-tumor behavior of the conjugate described in Example 8 in the absence of light, indicating that the conjugate molecule has potential immune anti-tumor activity.

### Example 9

The biliverdin-QQKFQFQFEQQ conjugate was prepared from biliverdin molecule and QQKFQFQFEQQ according to the above chemical synthesis method. A certain amount of conjugate was weighd, after being pre-dissolved in trace DMSO solution, the conjugate was directly dissolved in PBS solution, filtration sterilization was conducted after stirring and dissolving, and the pH value was adjusted to neutral. According to the standard tumor mouse modeling method, C57BL/6 mouse model was established, and the mouse colon cancer cells ct-26 were inoculated subcutaneously, and then these mice were fed in SPF environment. The tumor growth was observed at any time, and relevant experiments were carried out after the average tumor volume reached about 80-100 mm³. The mice were divided into the following four groups: blank group (normal saline), conjugate group (no light group) and conjugate group (light group), with 10 mice in each group. These mice were administered once on the 1st, 3rd, 8th and 12th day with the administration concentration being 2 mg kg⁻¹. Wherein, the mice of the conjugate (light group) were irradiated with laser once 4 hours after administration on the first day, and the parameters were as follows: the laser intensity was 0.5 W/cm² and the laser wavelength was 808 nm. The tumor inhibition of mice during the whole treatment cycle (the cycle is 45 days) was monitored. On the 29th day after the conjugate (light group) treatment, all the mice tumors were cleared, and the recurrence behavior of mice tumors was monitored from the 30th day to the 45th day. FIG. 9 shows the tumor inhibition curve (a) and recurrence curve (b) of the conjugate described in Example 9, which shows that the conjugate has a good therapeutic effect of tumor “photothermal immunotherapy” and can effectively prevent tumor recurrence.

### Example 10

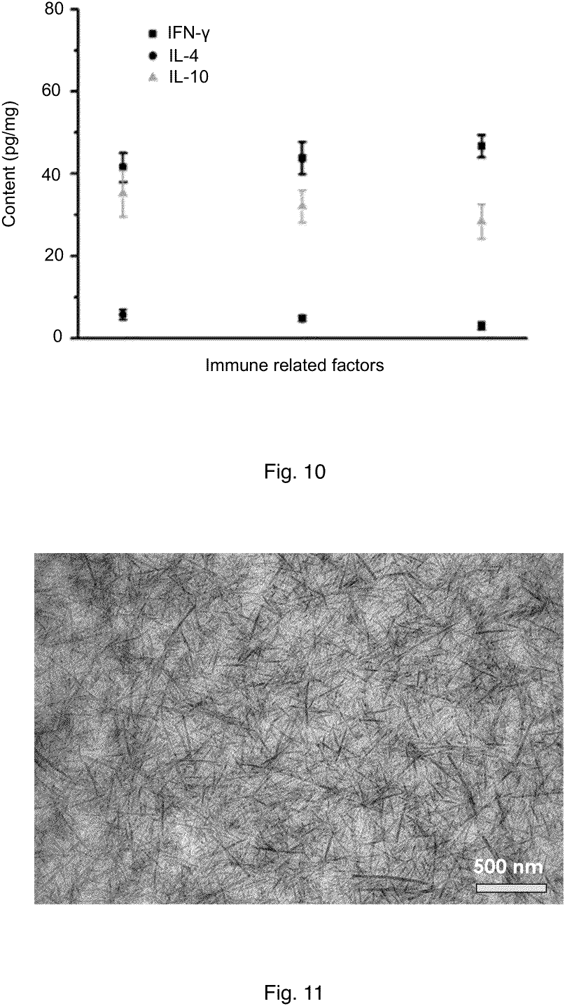

Firstly, biliverdin-Ga metal complex was chemically synthesized by biliverdin and excess gadolinium chloride hexahydrate, and the biliverdin-Ga-FKFEFKFE conjugate was prepared from biliverdin-Ga metal complex and FKFEFKFE according to the aforementioned chemical synthesis method. A certain amount of conjugate was weighed, after being pre-dissolved in trace DMSO solution, the conjugate was directly dissolved in PBS solution. Filtration sterilization was conducted after stirring and dissolving, and the pH value was adjusted to neutral. According to the standard tumor mouse modeling method, the in-situ model of pancreatic cancer Pan02 in C57BL/6 mouse was established, and then these mice were fed in SPF environment, and the tumor growth was observed at any time, and relevant experiments were carried out after the average tumor volume reached about 80-100 mm³. The mice were divided into the following four groups: blank group (normal saline), conjugate group (no light group) and conjugate group (light group), with 10 mice in each group. The mice were administered once on the 1st, 3rd, 8th and 12th day with the administration concentration of 4 mg kg⁻¹. Wherein, the mice of the conjugate (light group) were irradiated with laser once 4 hours after the first day of administration, and the parameters were as follows: the laser intensity was 0.5 W/cm² and the laser wavelength was 730 nm. On the 15th day, the mice were euthanized and the tumor tissues of each group were taken, and the contents of immune-related factors (comprising IFN-₇ with immune promoting effect and IL-4 and IL-10 with immune suppressing effect) in the supernatants of each group were measured by ELISA. FIG. 10 shows the content of immune-related factors described in Example 10, which indicates that the conjugate can up-regulate the organism’s immunity and down-regulate the immunosuppressive behavior with the presence and absence of light, indicating that the conjugate has a good therapeutic effect of tumor “photothermal immunotherapy”.

### Example 11

The biliverdin-LVVTPW conjugate was prepared from biliverdin molecule and LVVTPW according to the above chemical synthesis method. A certain amount of conjugate was weighted, after being pre-dissolved in trace DMSO solution,water was added to form the fiber dosage form of conjugate. And the concentration of the conjugate was 5 mg mL⁻¹. FIG. 11 is a transmission electron microscope picture of the conjugate molecular gel described in Example 11, showing a regular fiber network structure.

### Example 12

Firstly, biliverdin-Mn metal complex was chemically synthesized by biliverdin and excessive manganese chloride, and the biliverdin-Mn-ALCNTDSPL conjugate was prepared from the biliverdin-Mn metal complex and ALCNTDSPL according to the aforementioned chemical synthesis method. The conjugate was loaded into PLGA particles to prepare the conjugate emulsifier. According to the standard tumor mouse modeling method, the in-situ model of bladder cancer MB49 and MBT-2 in C57BL/6 mouse were estabblished, then these mice were fed in SPF environment, and the tumor growth was observed at any time, and relevant experiments were carried out after the average tumor volume reached about 80-100 mm³. The conjugate emulsifier was intravenously injected, after 6 hours, mice were placed under photoacoustic imager and nuclear magnetic resonance imager, and the photoacoustic signal and nuclear magnetic resonance signal intensity of tumor location were detected. FIG. 12 is a statistical chart of the results of the conjugate emulsion in Example 12 used for tumor imaging of mouse bladder cancer, proving the cancer diagnosis ability of the conjugate.

### Example 13

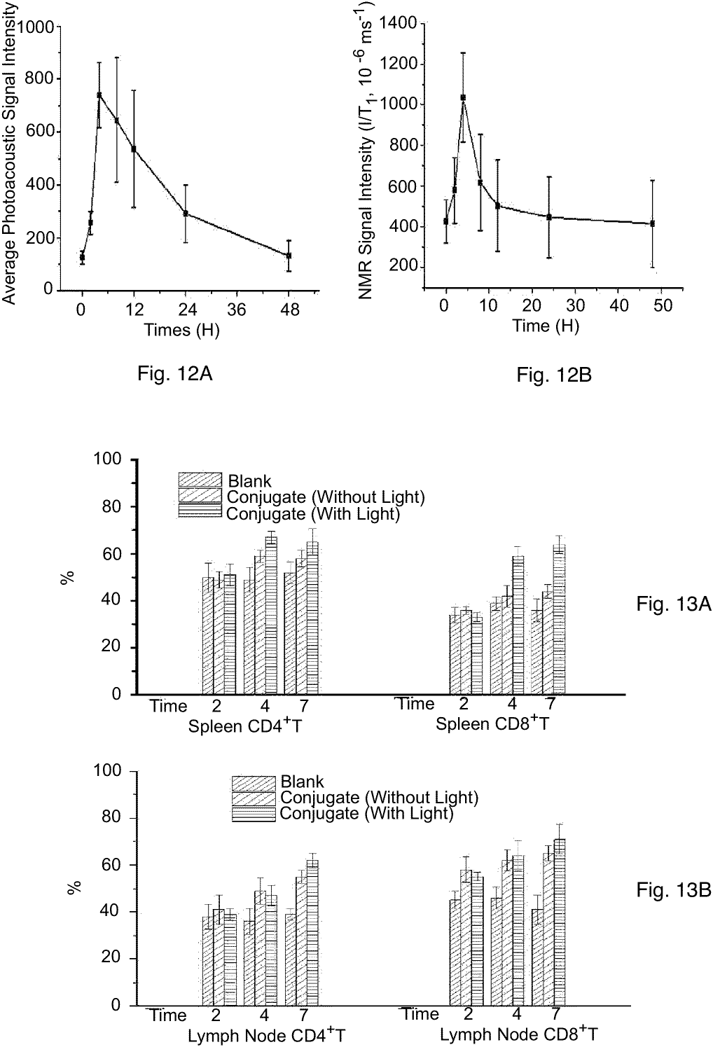

The biliverdin-EQLESIINFEKLTE conjugate was prepared from biliverdin molecule and EQLESIINFEKLTE according to the above chemical synthesis method. A certain amount of conjugate was weighed, after being pre-dissolved in trace DMSO solution, the conjugate was directly dissolved in PBS solution, after stirring and dissolving, filtration sterilization was conducted, and the pH value was adjusted to neutral. BALB/C cervical cancer U14 mouse model was established, and these mice were administered intraperitoneally with a concentration of 5 mg kg⁻¹. On the 2nd, 4th and 7th day after administration, the contents of CD4⁺T and CD8⁺T cells in spleen and draining lymph nodes of mice were detected by immunofluorescence staining and flow cytometry. FIG. 13 is the curve of CD4⁺T and CD8⁺T cells in spleen and draining lymph nodes of mice under the action of the conjugate described in Example 13, showing that the conjugate has immune effect and “photothermal immunotherapy” effect.

### Example 14

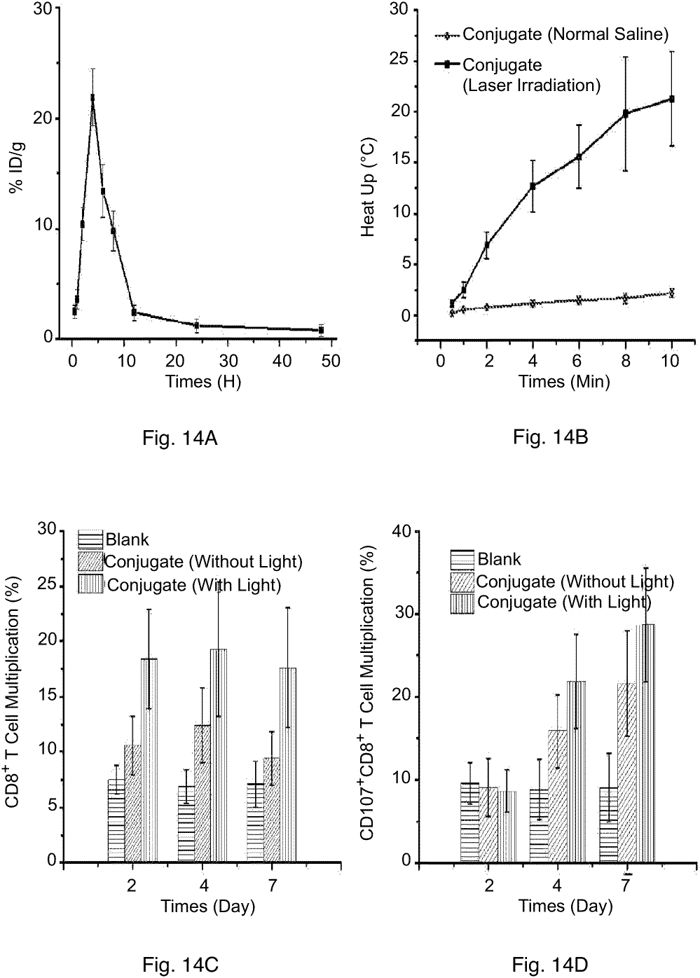

Firstly, the biliverdin was incubated with excess ⁹⁹mTc to obtain radiolabeled biliverdin, and the biliverdin-⁹⁹mTc-ISQAVHAAHAEEINEAGR conjugate was prepared from biliverdin⁻⁹⁹mTc and ISQAVHAAHAEEINEAGR according to the aforementioned chemical synthesis method. A certain amount of conjugate was weighed, after being pre-dissolved in trace DMSO solution, the conjugate was directly dissolve in PBS solution, after stirring and dissolving, filtration sterilization was conducted, and the pH value was adjusted to neutral. The conjugate was injected into tumor model mice (BALB/C, mouse breast tumor cell C127, the initial tumor volume was about 100 mm³) by intravenous injection, and its accumulation at the tumor site was monitored by single photon emission computed tomography. It was found that at the 4th hour after administration, the conjugate showed the clearest image at the tumor site, and the accumulated amount reached the highest value, which provided a window for tumor treatment. Under this time window, the tumor location was irradiated with laser (laser wavelength was 730 nm, power was 0.2 W/cm²), and the temperature change of the tumor location was monitored by near infrared imaging equipment. The expression of CD8⁺T cells and CD 107 molecules on the surface of CD8⁺T cells in mouse tumors were monitored by fluorescent immunostaining method, and the immune effect was evaluated. With regard to the conjugate described in Example 14, FIG. 14 shows the tumor radionuclide imaging results (a), in-situ tumor temperature rise (b), and the expression of CD8⁺T cells (c) and CD107 molecules on the surface of CD8⁺T cells (d) in mouse tumors, which proves that the conjugate can be used for cancer diagnosis and tumor “photothermal immunotherapy”.

### Example 15

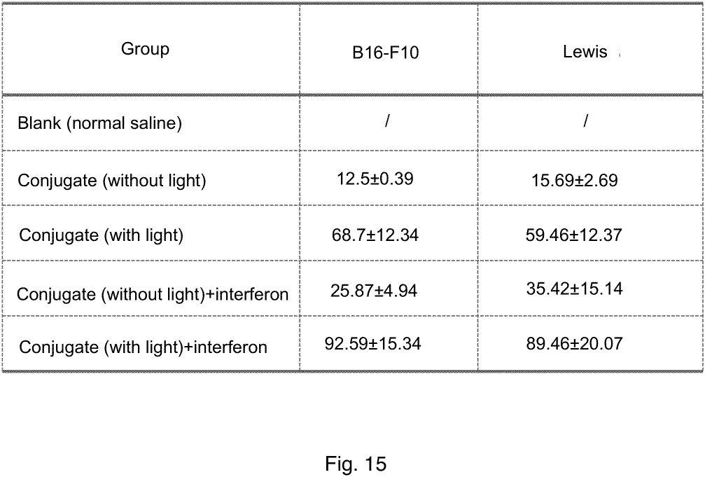

The biliverdin-PDRAHYNI conjugate was prepared from biliverdin molecule and PDRAHYNI according to the above chemical synthesis method. A certain amount of conjugate was weighed, after being pre-dissolved in a trace amount of organic solution, the conjugate was directly dissolved in PBS solution, after stirring and dissolving, filtration sterilization was conducted, and the pH value was adjusted to neutral. The C57BL/6 mouse model of skin melanoma B 16-F 10 and the C57BL/6 mouse model of lung cancer Lewis were established, and the combination of “photothermal immunotherapy” and immunotherapy was carried out. The administration concentration of the conjugate was 3 mg kg⁻¹, and the administration dosage of the immune drug interferon was 20U/ mouse. The administration window was that the tumor growth entered the logarithmic phase, and the initial volume was 300 mm³, and the inhibition of tumor was monitored. FIG. 15 shows the combined inhibitory effect of the conjugate of Example 15 combined with immune preparation on B16-F10 tumor and Lewis tumor. The results show that the combination of “photothermal immunity” and immunotherapy significantly enhances the anti-tumor effect.

### Example 16

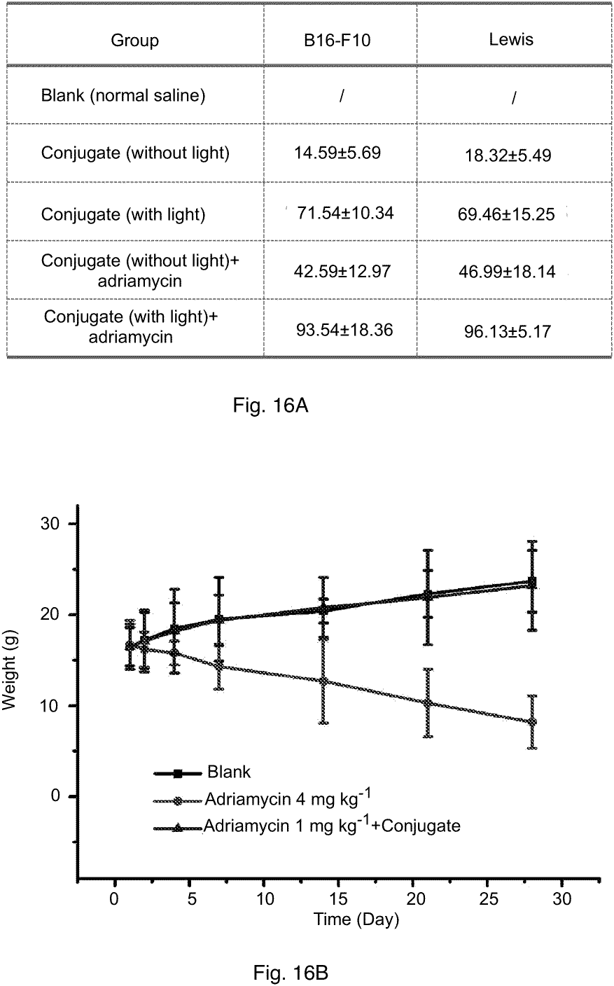

The biliverdin-MI,LAVLYCL conjugate was prepared from biliverdin molecule and MLLAVLYCL according to the above chemical synthesis method. A certain amount of conjugate was weighed, after being pre-dissolved in a trace amount of organic solution, the conjugate was directly dissolved in PBS solution. After stirring and dissolving, filtration sterilization was conducted, and the pH value was adjusted to neutral. The C57BL/6 mouse model of skin melanoma B16-F10 and the C57BL/6 mouse model of lung cancer Lewis were established, and the combination of “photo-immunotherapy” and chemotherapy was carried out. The administration concentration of the conjugate was 3 mg kg⁻¹, and the administration concentration of the chemotherapeutic drug doxorubicin was 1 mg kg⁻¹. The administration window was that the tumor growth entered the logarithmic phase, and the initial volume was 400 mm³, and the inhibition of tumor was monitored. FIG. 16 shows the combined inhibitory effect of the conjugate of Example 16 combined with low-dose chemotherapy drugs on B 16-F10 tumor and Lewis tumor, the results show that the combination of “photothermal immunity” and chemotherapy significantly enhance the anti-tumor effect (a) and effectively reduce the influence on the weight of mice.

### Example 17

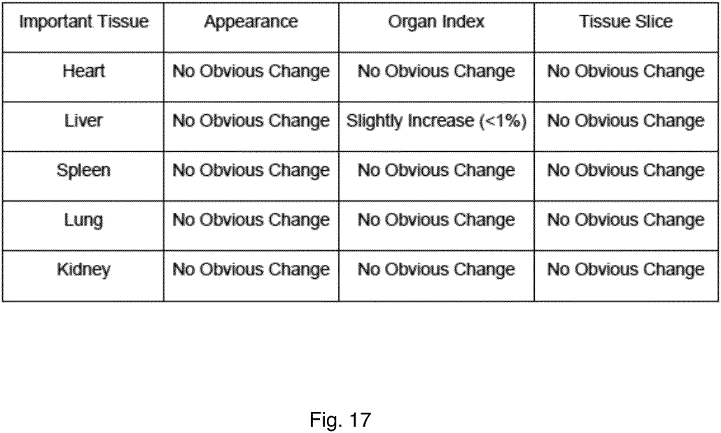

Firstly, biliverdin-Tb metal complex was chemically synthesized by biliverdin and excess terbium trichloride hexahydrate, and then biliverdin-Tb-VHFFKNIVTPTP conjugate was prepared from the biliverdin-Tb metal complex and VHFFKNIVTPTP according to the aforementioned chemical synthesis method. A certain amount of conjugate was weighed and directly dissolved in PBS solution, after stirring and dissolving, filtration sterilization was conducted, and the pH value was adjusted to neutral. The C57BL/6 mouse model of cutaneous melanoma B 16-F 10 was established, these mice were administered intravenously every other day for 5 times, and the dosage was 2 mg kg⁻¹. After 30 days, the tissues of a mouse were taken, the indexes of main organs were measured, and the biological safety of the conjugate was evaluated. FIG. 17 shows the influence of the conjugate described in Example 17 on important organs, and the result shows that it does not cause serious damage to important organs.

The applicant declares that the detailed method of the present disclosure is illustrated by the above-mentioned embodiments, but the present disclosure is not limited to the above-mentioned detailed method, that is, it does not mean that the present disclosure must be implemented by the above-mentioned detailed method. It should be clear to those skilled in the technical field that any improvement of the disclosure, equivalent substitution of raw materials of the product of the disclosure, addition of auxiliary ingredients, selection of specific methods, etc. all fall within the scope of protection and disclosure of the invention.

1. An immunologically active peptide-biliverdin conjugate, wherein the structure of the conjugate follows formula i, formula ii or formula iii, and salts, isomers and derivatives thereof that do not affect the pharmaceutical function; 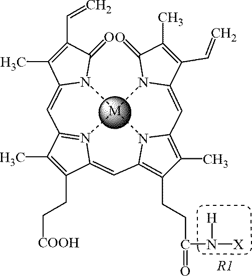 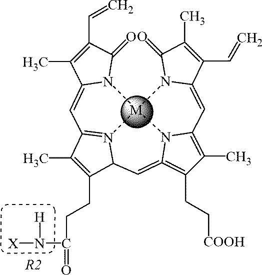 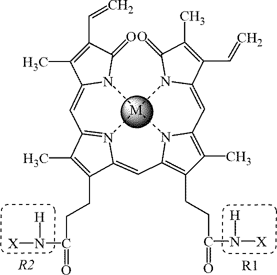
wherein
M is selected from the following nonmetallic atoms or ions of nonmetallic elements: H, Si, P; or metallic atoms or ions of metallic elements: Mg, Al, Cr, Mn, Fe, Co, Ni, Cu, Zn, Ga, Ge, Y, Ru, Rh, Pd, In, Sn, Pt, Au, Eu, Gd, Tb, Dy, Er, Yb, Lu, Tc, Tl; and radioisotopes and non-radioactive isotopes thereof; the number of M is 1-4;
R₁ and R₂ respectively represent an active peptide with the function of immunoregulation; and 1) the amino acid sequences of R₁ and R₂ are respectively any one of any group of X₁-X₂₂; or 2) R₁ and R₂ are respectively peptides or proteins comprising any sequences of the above; or derivatives of any sequences of the above; or amino acids, peptides or protein with similar functions of the above:
X₁: Ovalbumin peptide: SIINFEKL (8) (SEQ ID NO: 1), EQLESIINFEKLTE (14) (SEQ ID NO: 2), ISQAVHAAHAEINEAGR (17) (SEQ ID NO: 3);
X₂: HPV16 E7 peptide: PDRAHYNI (8) (SEQ ID NO: 4), TLGIVCPI (8) (SEQ ID NO: 5), RAHYNIVTF (9) (SEQ ID NO: 6), YMLDLQPETT (10) (SEQ ID NO: 7), GQAEPDRAHYNIVTF (15) (SEQ ID NO: 8);
X₃: NYSKPTDRQYHF (12) (SEQ ID NO: 9), KHAHHTHNLRLP (12) (SEQ ID NO: 10);
X₄: HVIHEGTVVI (10) (SEQ ID NO: 11), HVVHEGTVVI (10) (SEQ ID NO: 12);
X₅: KVPRNQDWL (9) (SEQ ID NO: 13), FLWGPRALV (9) (SEQ ID NO: 14);
X₆: YLEPGPVTA (9) (SEQ ID NO: 15), IMDQVPFSV (9) (SEQ ID NO: 16);
X₇: MLLAVLYCL (9) (SEQ ID NO: 17), YMDGTMSQV (9) (SEQ ID NO: 18);
X₈: TKPR (4) (SEQ ID NO: 19);
X₉: GQPR (4) (SEQ ID NO: 20);
X₁₀: CAPE (4) (SEQ ID NO: 21);
X₁₁: RKEVY (5) (SEQ ID NO: 22);
X₁₂: RKDVY (5) (SEQ ID NO: 23);
X₁₃: LVVTPW (6) (SEQ ID NO: 24);
X₁₄: FLGFPT (6) (SEQ ID NO: 25);
X₁₅: PDRAHYNI (8) (SEQ ID NO: 26);
X₁₆: FKFEFKFE (8) (SEQ ID NO: 27);
X₁₇: ALCNTDSPL (9) (SEQ ID NO: 28);
X₁₈: KIFGSLAFL (9) (SEQ ID NO: 29);
X₁₉: KTKCKFLKKC (10) (SEQ ID NO: 30);
X₂₀: QQKFQFQFEQQ (11) (SEQ ID NO: 31);
X₂₁: PLYKKIIKKLLES (13) (SEQ ID NO: 32);
X₂₂: HSLGKWLGHPDKF (13) (SEQ ID NO: 33);
X₂₃: VHFFKNIVTPRTP (13) (SEQ ID NO: 34);
X₂₄: EIIVTHFPFDEQNCSMK (17) (SEQ ID NO: 35);
X₂₅: (SNTSESF)2KFRVTQ-LAPKQIKE-NH₂ (29) (SEQ ID NO: 36).

2. The conjugate according to claim 1, wherein R₁ and R₂ are the same or different.

3. The conjugate according to claim 1
wherein the derivative of R₁ and R₂ is a peptide molecule or a key molecule fragment thereof modified by phenyl, benzyloxycarbonyl, tert-butoxycarbonyl, beta-naphthylamido, N-(3-indolacetyl) or N-fluorene methoxycarbonyl groups.

4. A preparation or dosage form derived from the conjugate according to claim 1, wherein the
preparation or dosage form system is formed by chemical bonding, physical adsorption, loading or wrapping; or comprising assemblies, polymers or aggregates formed by weak intermolecular interaction;
wherein, the preparation or dosage form comprises solution, emulsion, suspension, tablet, gel or patch.

5. A method for preparing the conjugate according to claim 1, comprising the following steps:
(1)M is H:
a. adding biliverdin, 1-ethyl-(3-dimethylaminopropyl) carbodiimide hydrochloride (EDC•HCl), N-hydroxysuccinimide (NHS) and anhydrous dimethylformamide (DMF) into a reactor sequentially, and mixing uniformly;
the concentration of the biliverdin is 0.1-500 mM, preferably, the concentration is 1-100 mM;
the concentration of the EDC•HCl is 0.1-1000 mM, preferably, the concentration is 1-200 mM;
the concentration of NHS is 0.1-1000 mM, preferably, the concentration is 1-200 mM;
the mass concentration ratio of biliverdin, EDC•HCl and NHS is 1: 1: 0.5-1: 20: 20, preferably, the mass concentration ratio is 1: 1: 0.8-1: 5: 10;
b. stirring the mixture obtained in step (a) at room temperature in the dark for 12-48 h, preferably 12-24 h;
c. adding water to the mixture obtained in step (b) while stirring, and collecting precipitate;
d. adding anhydrous DMF into the precipitate obtained in step (c), wherein the mass ratio of the precipitate to DMF is 1:100, preferably 1: 5;
e. adding immunologically active peptide and anhydrous triethylamine into the anhydrous DMF solution of the precipitate obtained in step (d), and stirring at room temperature in the dark;
the concentration of the immunologically active peptide is 0.01-2000 mM, preferably, the concentration is 0.1-500 mM;
the concentration of anhydrous triethylamine is 0.01-4000 mM, preferably, the concentration is 0.1-1,000 mm;
the stirring time is 4-96 h, preferably 12-24 h;
f. adjusting the pH value of the mixed solution obtained in step (e) to 3.5-7.5, preferably, the pH value is 4.0-6.0;
the pH value is adjusted by adding alkaline substances or acidic substances:
preferably, the alkaline substance is at least one selected from the group consisting of sodium hydroxide, potassium hydroxide and sodium carbonate;
preferably, the acidic substance is at least one selected from the group consisting of hydrochloric acid, sulfuric acid and nitric acid;
the pH value can also be adjusted by an aqueous solution dialysis method;
g. collecting the precipitate in step (f) and purifying the precipitate by size exclusion chromatography;
h. recrystallizing the substance obtained in step (g) to obtain a pure molecular conjugate;
(2)M is a metal atom or ion except H:
i. dissolving biliverdin and excess metal acetate in methanol, wherein the mass concentration ratio of biliverdin and metal salt is 1: 1-1: 100, preferably 1: 2-1: 20;
j. stirring the methanol solution in step (i) for 4 hours at a certain temperature ranging from 20° C. to 60° C., preferably from 35° C. to 60° C.;
k. removing the solvent from the solution obtained in the step (j) by rotary evaporation to obtain a solid;
1. purifying the solid obtained in the step (k) by a reversed-phase chromatographic column to obtain the biliverdin-metal complex;
m. according to steps a to h, the synthesis of biliverdin-metal complex-immunologically active peptide conjugate is achieved; or
(3)M is a nonmetallic atom or ion except H:
n. dissolving biliverdin and nonmetallic halide or acyl chloride in organic solvent pyridine or DMF, wherein the mass concentration ratio of biliverdin and nonmetallic halide or acyl chloride is 1: 1-1: 100, preferably 1: 2-1: 20;
o. stirring the mixed solution obtained in step (n) at a certain temperature in the dark, with the temperature ranging from 20 to 100° C., preferably from 35 to 65° C.; wherein the reaction time is 2-8 h, preferably 4-6 h;
p. removing the solvent from the solution obtained in the step (o) by rotary evaporation;
q. purifying the solid obtained in the step (p) by a reversed-phase chromatographic column to obtain the biliverdin-nonmetal complex;
r. according to steps a to h, the synthesis of biliverdin-nonmetal complex-immunologically active peptide conjugate is achieved.

6. A photothermal immune anti-tumor method, comprising administering a therapeutically effective amount of the conjugate according to claim 1 to a subject in need thereof.

7. The method according to claim 6, wherein the anti-tumor method has simultaneous functions of tumor ablation, immune regulation and tumor inflammatory microenvironment regulation.

8. A method of nuclear magnetic resonance imaging for a tumor,comprising administering a therapeutically effective amount of the conjugate according to claim 1 to a subject in need thereof, wherein M is at least one selected from the group consisting of the following atoms or ions: Mn, Fe, Cu, Eu, Gd and Dy.

9. A method of radionuclide imaging for tumor detection, comprising administering a therapeutically effective amount of the conjugate according to claim 1 to a subject in need thereof, wherein M is at least one selected from the group consisting of the following atoms or ions: ^{64,67}Cu, ⁹⁹mTc, ¹⁹⁵Pt, ^{67,68}Gd, ²⁰¹T1, ⁶⁰Co, ¹¹¹In and ⁵¹Cr.

10. A method of photoacoustic imaging of a tumor, comprising administering a therapeutically effective amount of the conjugate according to claim 1 to a subject in need thereof, wherein M is at least one selected from the group consisting of the following atoms or ions: H and Zn.

11. The conjugate according to claim 1, wherein the active site of the immunologically active peptide is at the non-N end, and the inactive terminal is condensed with the C end of biliverdin by peptide bond.

12. The conjugate according to claim 1, wherein the immunologically active peptide has the following amino acid sequence; or comprises the following sequence; or is a derivative of the following sequences; or is an amino acid, peptide or protein with similar functions of the following sequences:
X₁: SIINFEKL (8) (SEQ ID NO: 1);
X₃: NYSKPTDRQYHF (12) (SEQ ID NO: 9);
X₅: FLWGPRALV (9) (SEQ ID NO: 14);
X₆: YLEPGPVTA (9) (SEQ ID NO: 15), IMDQVPFSV (9) (SEQ ID NO: 16);
X₇: YMDGTMSQV (9) (SEQ ID NO: 18);
X₁₅: PDRAHYNI (8) (SEQ ID NO: 26);
X₁₈: KIFGSLAFL (9) (SEQ ID NO: 29);
X₂₃: VHFFKNIVTPRTP (13) (SEQ ID NO: 34).

13. The method according to claim 6, wherein the tumor is a primary tumor or a metastatic tumor, and is a single tumor or multiple tumors selected from the group consisting of brain cancer, head and neck cancer, esophageal cancer, breast cancer, lung cancer, stomach cancer, liver cancer, colon cancer, pancreatic cancer, lymphoma, melanoma, ovarian cancer, cervical cancer, prostate cancer and bladder cancer;
wherein preferably the tumor is a superficial tumor or a tumor with high surgical risk; wherein the superficial tumor or the tumor with high surgical risk comprises head and neck cancer, breast cancer, melanoma, cervical cancer, prostate cancer, or pancreatic cancer.

14. The method according to claim 8, wherein the tumor is a primary tumor or a metastatic tumor, and is a single tumor or multiple tumors selected from the group consisting of brain cancer, head and neck cancer, esophageal cancer, breast cancer, lung cancer, stomach cancer, liver cancer, colon cancer, pancreatic cancer, lymphoma, melanoma, ovarian cancer, cervical cancer, prostate cancer and bladder cancer;
wherein preferably the tumor is a superficial tumor or a tumor with high surgical risk; wherein the superficial tumor or the tumor with high surgical risk comprises head and neck cancer, breast cancer, melanoma, cervical cancer, prostate cancer, or pancreatic cancer.

15. The method according to claim 9, wherein the tumor is a primary tumor or a metastatic tumor, and is a single tumor or multiple tumors selected from the group consisting of brain cancer, head and neck cancer, esophageal cancer, breast cancer, lung cancer, stomach cancer, liver cancer, colon cancer, pancreatic cancer, lymphoma, melanoma, ovarian cancer, cervical cancer, prostate cancer and bladder cancer;
wherein preferably the tumor is a superficial tumor or a tumor with high surgical risk; wherein the superficial tumor or the tumor with high surgical risk comprises head and neck cancer, breast cancer, melanoma, cervical cancer, prostate cancer, or pancreatic cancer.

16. The method according to claim 10, wherein the tumor is a primary tumor or a metastatic tumor, and is a single tumor or multiple tumors selected from the group consisting of brain cancer, head and neck cancer, esophageal cancer, breast cancer, lung cancer, stomach cancer, liver cancer, colon cancer, pancreatic cancer, lymphoma, melanoma, ovarian cancer, cervical cancer, prostate cancer and bladder cancer;
wherein preferably the tumor is a superficial tumor or a tumor with high surgical risk; wherein the superficial tumor or the tumor with high surgical risk comprises head and neck cancer, breast cancer, melanoma, cervical cancer, prostate cancer, or pancreatic cancer.

17. The method according to claim 6, wherein the photothermal immune anti-tumor method is combined with tumor therapy of surgery, chemotherapy, radiotherapy or immunotherapy.

18. The method according to claim 17, wherein drugs for chemotherapy comprise at least one drug selected from the group consisting of cisplatin, carboplatin, nedaplatin, oxaliplatin, lobaplatin, carmustine, lomustine, smoustine, nimustine, methotrexate, pemetrexed, nolatrexed, raltitrexed, fluorouracil, capecitabine, gemcitabine, ancitabine, cytarabine, tegafur, fluorouridine, doxifluridine, youfuding, vinblastine, vincristine, vinblastine, vindesine, vinorelbine, paclitaxel, docetaxel, albumin-bound paclitaxel, camptothecin, irinotecan, topotecan, rubitecan, doxorubicin, epirubicin, pirarubicin, amide, isocyclophosphamide, etoposide, and derivatives thereof.

19. The method according to claim 17, wherein drugs for immunotherapy comprise at least one selected from the group consisting of antibodies, cytokines, molecular vaccines, cell vaccines, biological response regulators, immune inhibitor, and monomer components of traditional Chinese medicine.

20. The method according to claim 17, wherein drugs for immunotherapy comprise at least one selected from the group consisting of thymic factor, indoleamine 2,3-dioxygenase inhibitor, interferon and interleukin.


SEQUENCE LISTING (36 sequences)

SEQ ID NO: 1 - protein, 8 aa, Artificial Sequence
  Active immunoregulatory ovalbumin peptide sequence
SIINFEKL

SEQ ID NO: 2 - protein, 14 aa, Artificial Sequence
  Active immunoregulatory ovalbumin peptide sequence
EQLESIINFEKLTE

SEQ ID NO: 3 - protein, 17 aa, Artificial Sequence
  Active immunoregulatory ovalbumin peptide sequence
ISQAVHAAHAEINEAGR

SEQ ID NO: 4 - protein, 8 aa, Artificial Sequence
  Active immunoregulatory HPV16 E7 peptide sequence
PDRAHYNI

SEQ ID NO: 5 - protein, 8 aa, Artificial Sequence
  Active immunoregulatory HPV16 E7 peptide sequence
TLGIVCPI

SEQ ID NO: 6 - protein, 9 aa, Artificial Sequence
  Active immunoregulatory HPV16 E7 peptide sequence
RAHYNIVTF

SEQ ID NO: 7 - protein, 10 aa, Artificial Sequence
  Active immunoregulatory HPV16 E7 peptide sequence
YMLDLQPETT

SEQ ID NO: 8 - protein, 15 aa, Artificial Sequence
  Active immunoregulatory HPV16 E7 peptide sequence
GQAEPDRAHYNIVTF

SEQ ID NO: 9 - protein, 12 aa, Artificial Sequence
  Active immunoregulatory peptide sequence
NYSKPTDRQYHF

SEQ ID NO: 10 - protein, 12 aa, Artificial Sequence
  Active immunoregulatory peptide sequence
KHAHHTHNLRLP

SEQ ID NO: 11 - protein, 10 aa, Artificial Sequence
  Active immunoregulatory peptide sequence
HVIHEGTVVI

SEQ ID NO: 12 - protein, 10 aa, Artificial Sequence
  Active immunoregulatory peptide sequence
HVVHEGTVVI

SEQ ID NO: 13 - protein, 9 aa, Artificial Sequence
  Active immunoregulatory peptide sequence
KVPRNQDWL

SEQ ID NO: 14 - protein, 9 aa, Artificial Sequence
  Active immunoregulatory peptide sequence
FLWGPRALV

SEQ ID NO: 15 - protein, 9 aa, Artificial Sequence
  Active immunoregulatory peptide sequence
YLEPGPVTA

SEQ ID NO: 16 - protein, 9 aa, Artificial Sequence
  Active immunoregulatory peptide sequence
IMDQVPFSV

SEQ ID NO: 17 - protein, 9 aa, Artificial Sequence
  Active immunoregulatory peptide sequence
MLLAVLYCL

SEQ ID NO: 18 - protein, 9 aa, Artificial Sequence
  Active immunoregulatory peptide sequence
YMDGTMSQV

SEQ ID NO: 19 - protein, 4 aa, Artificial Sequence
  Active immunoregulatory peptide sequence
TKPR

SEQ ID NO: 20 - protein, 4 aa, Artificial Sequence
  Active immunoregulatory peptide sequence
GQPR

SEQ ID NO: 21 - protein, 4 aa, Artificial Sequence
  Active immunoregulatory peptide sequence
CAPE

SEQ ID NO: 22 - protein, 5 aa, Artificial Sequence
  Active immunoregulatory peptide sequence
RKEVY

SEQ ID NO: 23 - protein, 5 aa, Artificial Sequence
  Active immunoregulatory peptide sequence
RKDVY

SEQ ID NO: 24 - protein, 6 aa, Artificial Sequence
  Active immunoregulatory peptide sequence
LVVTPW

SEQ ID NO: 25 - protein, 6 aa, Artificial Sequence
  Active immunoregulatory peptide sequence
FLGFPT

SEQ ID NO: 26 - protein, 8 aa, Artificial Sequence
  Active immunoregulatory peptide sequence
PDRAHYNI

SEQ ID NO: 27 - protein, 8 aa, Artificial Sequence
  Active immunoregulatory peptide sequence
FKFEFKFE

SEQ ID NO: 28 - protein, 9 aa, Artificial Sequence
  Active immunoregulatory peptide sequence
ALCNTDSPL

SEQ ID NO: 29 - protein, 9 aa, Artificial Sequence
  Active immunoregulatory peptide sequence
KIFGSLAFL

SEQ ID NO: 30 - protein, 10 aa, Artificial Sequence
  Active immunoregulatory peptide sequence
KTKCKFLKKC

SEQ ID NO: 31 - protein, 11 aa, Artificial Sequence
  Active immunoregulatory peptide sequence
QQKFQFQFEQQ

SEQ ID NO: 32 - protein, 13 aa, Artificial Sequence
  Active immunoregulatory peptide sequence
PLYKKIIKKLLES

SEQ ID NO: 33 - protein, 13 aa, Artificial Sequence
  Active immunoregulatory peptide sequence
HSLGKWLGHPDKF

SEQ ID NO: 34 - protein, 13 aa, Artificial Sequence
  Active immunoregulatory peptide sequence
VHFFKNIVTPRTP

SEQ ID NO: 35 - protein, 17 aa, Artificial Sequence
  Active immunoregulatory peptide sequence
EIIVTHFPFDEQNCSMK

SEQ ID NO: 36 - protein, 28 aa, Artificial Sequence
  Active immunoregulatory peptide sequence
SNTSESFSNTSESFKFRVTQLAPKQIKE